(19) United States
(12) Patent Application Publication
(10) Pub. No.: US 2004/0082069 A1
(43) Pub. Date: Apr. 29, 2004

(54) Systems and methods for estimating exposure temperatures and remaining operational life of high temperature components

(76) Inventors: Liang Jiang, Guilderland, NY (US); Ji-Cheng Zhao, Latham, NY (US); Lawrence Kool, Clifton Park, NY (US); Melvin Jackson, Niskayuna, NY (US); Canan Hardwicke, Niskayuna, NY (US); Ann Ritter, Niskayuna, NY (US); Ching-Pang Lee, Cincinnati, OH (US)

Correspondence Address: Dougherty, Clements & Hofer, Attn: Tracey R. Loughlin, 1901 Roxborough Road, Suite 300, Charlotte, NC, 28211, US

(21) Appl. No.: 10/280,215

(22) Filed: Oct. 25, 2002

(51) Int. Cl. G01N 33/00

(52) U.S. Cl. 436/2; 422/68.1

(57) ABSTRACT

Non-destructive systems and methods for temperature measurements are described so that the remaining operational life and accumulated damage of high temperature gas turbine components can be assessed. Alloy-based witness coupons and diffusion couple witness coupons are attached to, or directly applied onto, high temperature components so that they experience the same high temperature operation and shut down as the components themselves. The witness coupons are later removed from the components and analyzed, or are analyzed on the component, to determine the change to their microstructure, metallurgy, and/or diffusion characteristics. Since the time each component spends in operation is known, the operating temperatures of the components can be back-calculated from the microstructural, metallurgical, and/or diffusion characteristic changes of the witness coupons. Therefrom, the remaining operational life of the component can be assessed, as can the accumulated damage to the component.

### FIELD OF THE INVENTION

The present invention relates generally to systems and methods for estimating the temperatures experienced by a component, specifically, gas turbine hot-gas-path components. More specifically, the present invention relates to systems and methods that utilize analysis of the metallurgical changes of a material to estimate the temperatures that a high temperature gas turbine component has experienced, so that, if desired, the remaining operational life and accumulated damage of the component may be assessed therefrom.

### BACKGROUND OF THE INVENTION

Measuring the temperatures that a high temperature turbine component (i.e., hot-gas-path components such as blades, vanes and/or shrouds in a gas turbine) has experienced is very important so that turbine design can be verified, metallurgical changes can be estimated, the remaining operational life of the component can be estimated, inspection intervals can be optimized, and operational conditions can be regulated. Such information is of great importance to both aircraft and power generation industries. Components that work in high temperature environments are particularly susceptible to degradation, the extent of which depends on a number of factors, such as the creep rate, rupture stress, stress/strain amplitude of cyclic loading, corrosion and/or erosion rate, and thermal mechanical fatigue, among other things. In some cases, such as when exposed to high temperatures for prolonged periods of time, material undergoes metallurgical changes (i.e., chemistry, microstructure, etc.) that reduce the material's reliability and durability. The degree of effect that these factors may have depends on the operational working temperatures of the components. Therefore, the temperatures that are experienced by a component are an important parameter governing the life of such components, as is the time that is spent at these temperatures. As such, many life assessment procedures have been developed to estimate the remaining operational life of such components based on the operating temperatures that these components have experienced, and the time they have spent in operation.

Currently, there are both destructive and non-destructive systems and methods for estimating the temperatures that a gas turbine component has experienced during operation. Some destructive systems and methods involve cutting up the component itself so that the characteristic metallurgical changes in the component can be investigated, and the time-temperature relationship can be estimated therefrom. Such methods utilize calculations similar to the Larson-Miller relationship. For example, a relationship of a precipitation amount, a temperature, and a time of a characteristic phase may be obtained for a high temperature gas turbine part formed of nickel-based single crystal alloy, and the temperature and remaining operational life of this part may then be estimated from this relationship by investigating the microstructure of the part. Non-destructive systems and methods that have been used to estimate the temperatures that hot-gas-path components in gas turbines have experienced include using thermocouples, pyrometers, eddy current sensors and/or temperature probes, among other things.

These current systems and methods have significant drawbacks: 1) many require a laborious procedure; 2) many use a complex arrangement of sensors; 3) many are barely able to sustain long hours at the high temperatures that gas turbine components experience; 4) many are not resistant to the hostile environment (i.e., oxidation, corrosion) that gas turbine components experience; 5) many are destructive to the components themselves, and/or 6) many are not suitable for moving parts. Thus, there is a need for systems and methods that allow the temperatures that such gas turbine components experience to be measured or estimated more reliably, accurately, conveniently and easily. There is also a need for such systems and methods to allow the remaining operational life and/or accumulated damage of such gas turbine components to be assessed. There is yet a further need for such systems and methods to be non-destructive to the gas turbine components themselves. There is still a further need for such systems and methods to utilize witness coupons for estimating the temperatures that such gas turbine components experience. There is also a need for such systems and methods to utilize: 1) alloy-based witness coupons comprising at least one alloy comprising at least two phases and having the characteristic feature that the solubility of at least one element in one of the phases changes greatly with temperature changes, or 2) diffusion couple witness coupons comprising pure element couples such as rhodium-platinum, or alloy couples such as Co30Cr—Co20Cr10Al (weight %), or couples comprising combinations of pure elements and alloys such as Pt—Co30Cr (weight %). There is also a need for such systems and methods to estimate the temperatures that are experienced by such gas turbine components based on the metallurgical changes of the witness coupons. There is also a need for such systems and methods to estimate the temperatures that are experienced by such gas turbine components based on the diffusion characteristic changes of the witness coupons. Finally, there is a need for such systems and methods to be designed so they do not interfere with the aerodynamics and mechanical design of the gas turbine.

### SUMMARY OF THE INVENTION

Accordingly, the above-identified shortcomings of existing systems and methods are overcome by embodiments of the present invention. This invention relates to systems and methods for estimating the temperatures that a high temperature component (i.e., a gas turbine component) has experienced, so that the remaining operational life and accumulated damage of the component can be assessed. Embodiments of this invention comprise systems and methods that require no laborious procedure or complex arrangement of sensors, that are able to sustain long hours at the high temperatures that gas turbine components experience, that are resistant to the hostile environment that gas turbine components experience (i.e., that are oxidation and/or corrosion resistant), that are not destructive to the turbine components themselves, and/or that are suitable for moving parts. In some embodiments, the systems and methods of this invention may allow the temperatures that gas turbine components experience to be measured or estimated more reliably, accurately, conveniently and easily than currently possible. Embodiments of this invention may allow the remaining operational life and/or accumulated damage of such gas turbine components to be assessed. In embodiments, this invention may be non-destructive to the gas turbine components themselves. Furthermore, embodiments of this invention can be designed so they utilize witness coupons for estimating the temperatures that such gas turbine components experience. These witness coupons may comprise: I) alloy-based witness coupons comprising at least one alloy comprising at least two phases and having the characteristic feature that the solubility of at least one element in one of the phases changes greatly with temperature changes, or 2) diffusion couple witness coupons comprising pure element couples such as rhodium-platinum, or alloy couples such as Co30Cr—Co20Cr10Al (weight %), or couples comprising combinations of pure elements and alloys such as Pt—Co30Cr (weight %). The temperatures that gas turbine components experience may be estimated based on the metallurgical changes of these witness coupons. Alternatively, the temperatures that gas turbine components experience may be estimated based on the diffusion characteristic changes of these witness coupons. Finally, embodiments of this invention may be designed so they do not interfere with the aerodynamics and mechanical design of the gas turbine.

This invention comprises witness coupons that are useful for estimating the temperatures that high temperature gas turbine components have experienced. Since the time a component has spent in operation is a known parameter (or one that can be easily found out), these estimated temperatures may then be used to determine how much operational life remains for a given component, or to determine how much accumulated damage has occurred to the component. As used herein, the term “witness coupon” comprises two different types of witness coupons: alloy-based witness coupons and diffusion couple witness coupons. The alloy-based witness coupons comprise an alloy containing at least two phases, wherein the alloy has the characteristic feature that the solubility of at least one element in one of the phases changes greatly with temperature changes. Additionally, other characteristic features of the alloy, such as the chemistry, lattice parameter, phase fraction, and electrical or magnetic properties, may also change greatly according to various temperatures. The second type of witness coupons, the diffusion couple witness coupons, comprise pure element couples such as rhodium-platinum, or alloy couples such as Co30Cr—Co20Cr10Al (wt. %), or couples comprising a combination of pure elements and alloys such as Pt—Co30Cr (wt. %). The diffusion couple witness coupons have the characteristic feature that there is interdiffusion between the components of the diffusion couple (i.e., between the pure elements and/or the alloys). The interdiffusion process is a function of time and temperature, thus the changes of the characteristic properties of the diffusion couple witness coupons (i.e., interdiffusion distance, interdiffusion profile, chemistry at certain locations in the interdiffusion zone, and the corresponding electrical and magnetic properties) are also functions of time and temperature.

Typically, high temperature gas turbine components experience high operating temperatures for a given period of time, and are then shut down. The shut down process is similar to a quenching process, where the component is quickly cooled down from the high operating temperature. The witness coupons of this invention may be attached to a high temperature gas turbine component so as not to interfere with the aerodynamics or mechanical design of the component. In this manner, the witness coupon can experience the same high temperature operation and shut down as the component itself. These alloy-based witness coupons are designed so that the time spent at the high operating temperatures changes the microstructure of the alloy in the alloy-based witness coupon, and the fast cooling of the component and the alloy-based witness coupon during the shut down process preserves the high temperature microstructure in the alloy-based witness coupon. These diffusion couple witness coupons are designed so that the time spent at the high operating temperatures causes interdiffusion between the pure elements and/or the alloys, which is preserved after the component is shut down. Since the witness coupons experience the same operational and shut down temperatures as the component itself, the witness coupons carry information about the operating temperature of the component. These witness coupons can then be removed from the component and analyzed, or they can be analyzed while still on or attached to the component, to determine the operating temperatures the component was exposed to, and the remaining operational life of the component can be determined therefrom. The accumulated damage to the component may also be determined from the estimated operating temperatures of the component.

One embodiment of this invention comprises the witness coupons themselves. Other embodiments of this invention comprise systems and methods for characterizing metallurgical changes and/or diffusion characteristic changes and estimating the temperatures that a high temperature component has experienced so that the remaining operational life and/or accumulated damage of the high temperature component can be assessed. Yet other embodiments of this invention comprise methods of making and implementing the witness coupons.

This invention has all the advantages of existing systems and methods, but it is non-destructive to the gas turbine components themselves, and allows the remaining operational life and/or accumulated damage of gas turbine components to be more easily, accurately, conveniently and reliably assessed.

Further features, aspects and advantages of the present invention will be more readily apparent to those skilled in the art during the course of the following description, wherein references are made to the accompanying figures which illustrate some preferred forms of the present invention, and wherein like characters of reference designate like parts throughout the drawings.

### DESCRIPTION OF THE DRAWINGS

The systems and methods of the present invention are described herein below with reference to various figures, in which:

FIG. 1 is a photograph showing several witness coupons of the present invention attached to the tip caps of turbine blades; and

FIG. 2 is a scanning electron microscopy (SEM) micrograph showing the interdiffusion of Rh and Pt in one embodiment of a diffusion couple witness coupon of this invention.

### DETAILED DESCRIPTION OF THE INVENTION

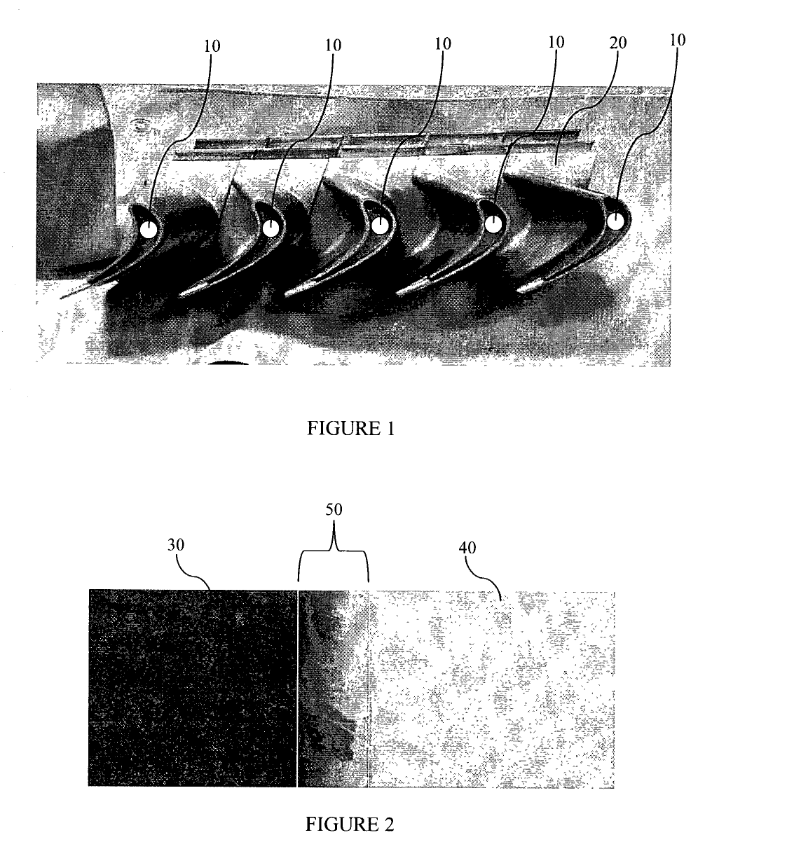

For the purposes of promoting an understanding of the invention, reference will now be made to some preferred embodiments of the present invention as illustrated in FIGS. 1-2, and specific language used to describe the same. The terminology used herein is for the purpose of description, not limitation. Specific structural and functional details disclosed herein are not to be interpreted as limiting, but merely as a basis for the claims as a representative basis for teaching one skilled in the art to variously employ the present invention. Any modifications or variations in the depicted systems and methods, and such further applications of the principles of the invention as illustrated herein, as would normally occur to one skilled in the art, are considered to be within the spirit of this invention.

The present invention comprises witness coupons that are useful for estimating the temperatures that high temperature gas turbine components have experienced. Since the times these components have spent in operation is a known parameter, the temperatures these components have experienced can be estimated, and may then be used to determine how much operational life remains for a given component. These estimated temperatures may also be used to determine how much damage has been accumulated to a given component. The alloy-based witness coupons comprise an alloy containing at least two phases, wherein the alloy has the characteristic feature that the solubility of at least one element in one of the phases changes greatly with temperature changes. Additionally, other characteristic features, such as the chemistry, lattice parameter, phase fraction, hardness/modulus, and electrical or magnetic properties, also change greatly in the alloy according to various temperatures.

The alloy-based witness coupons may be made in several different manners. A melting-solidification process may be used to make these alloy-based witness coupons, where the alloy is first melted and then solidified. These alloy-based witness coupons may also be made by a deposition process, such as by sputtering, thermal spraying, ion plasma deposition, or the like. Another way these alloy-based witness coupons may be made is by a powder deposition and sintering process, where the alloy and/or pure element mixture is deposited onto the area of interest on the component by direct-writing of inks containing metal powders, paste, or tape, then the mixture is consolidated by high temperature sintering. These powder mixtures may also be processed by laser or electron cladding processes.

One exemplary method of making an alloy-based witness coupon comprises: casting an alloy ingot of an alloy-based witness coupon using induction melting, arc melting, or the like; performing high temperature annealing to homogenize the alloy ingot; and sectioning the alloy ingot into appropriate sized alloy-based witness coupons that can be attached to a high temperature component.

Another exemplary method of making an alloy-based witness coupon comprises: depositing a layer of a material onto a high temperature component using a direct-writing method, physical vapor deposition, chemical vapor deposition, or the like; and depositing additional layers of the material onto the high temperature component as desired to make an alloy-based witness coupon.

The diffusion couple witness coupons may be made by electron beam welding the edges of metal foils, followed by hot isostatic pressing (HIP) at a predetermined temperature for a predetermined amount of time. These diffusion couple witness coupons may also be made by a deposition process, such as by sputtering, thermal spraying, ion plasma deposition, or the like. Another way these diffusion couple witness coupons may be made is by a powder deposition and sintering process, where the alloy and/or pure element mixture is deposited onto the area of interest on the component by direct-writing of inks containing metal powders, paste, or tape, then the mixture is consolidated by high temperature sintering. These powder mixtures may also be processed by laser or electron cladding processes.

One exemplary method of making a diffusion couple witness coupon comprises: pressing thin foils of metals together using cold pressing, cold isostatic pressing, hot isostatic pressing, or the like to make diffusion couple sheets; and then sectioning the diffusion couple sheets into appropriate sized diffusion couple witness coupons that can be attached to a high temperature component.

These manners of making the alloy-based witness coupons and the diffusion couple witness coupons are meant to be exemplary, not limiting, examples of how these witness coupons can be made. Many other methods of making these witness coupons are also possible, as will be recognized by those skilled in the art.

Before use, relationships of temperature-specific characteristics of a witness coupon may first need to be established and catalogued. For example, the chemistry, lattice parameter/phase fraction, hardness/modulus, electric properties and/or magnetic properties, and/or the diffusion distances at various temperatures, for a witness coupon could be established and calibrated corresponding to the applicable operational temperature range of gas turbines. Any suitable methods may be utilized to measure or determine the temperature-specific characteristics of the witness coupons.

For example, the chemistry of the alloy-based witness coupons may be measured or determined by electron microprobe analysis using either wavelength dispersive spectroscopy (WDS) or energy dispersive spectroscopy (EDS), x-ray fluorescence, laser plasma spectroscopy, or the like. The lattice parameter/phase fraction of the alloy-based witness coupons may be measured or determined by high energy x-ray, neutron diffraction analysis, image analysis integrating optical and/or electron microscopy, or the like. The hardness/modulus of either the alloy-based witness coupons or the diffusion couple witness coupons may be measured or determined by nanoindentation, microhardness testing, ultrasonic modulus measurement techniques, or the like. The electric properties (in terms of resistivity and/or conductivity) of either the alloy-based witness coupons or the diffusion couple witness coupons may be measured or determined by eddy current probe. The magnetic properties (in terms of magnetic field) of either the alloy-based witness coupons or the diffusion couple witness coupons can be measured or determined by eddy current probe. The diffusion characteristics (i.e., diffusion distances at various temperatures) of the diffusion couple witness coupons can be measured or determined by electron microprobe analysis using either wavelength dispersive spectroscopy (WDS) or energy dispersive spectroscopy (EDS), or nanoindentation. Finally, the surface micro-voltage of either the alloy-based witness coupons or the diffusion couple witness coupons may be measured by thermoelectric unit measurements.

Once the operational conditions are identified, and the relationships of the temperature-specific characteristics of a witness coupon are established, a witness coupon may then be attached to, or applied directly onto, a high temperature gas turbine component so as not to interfere with the aerodynamics or mechanical design of the component. For example, as shown in FIG. 1, witness coupons 10 may be attached to the tip caps of a gas turbine blade 20 so that blade temperatures can be measured and/or estimated. While the witness coupons 10 depicted here are circular, any other suitable shape is also feasible. Witness coupons 10 could also be attached to any other suitable component or location where the temperature condition during operation needs to be diagnosed or evaluated. Witness coupons 10 could also be applied as a coating, directly onto the surface of the component. In either manner, the witness coupons can experience the same high temperature operation and shut down cycles as the component itself.

Witness coupons may be attached to any suitable desired location on a turbine component in any suitable manner, such as by diffusion bonding, electron beam welding, laser welding, brazing, spraying, sputtering, ion plasma processing, suitable mechanical attachment means, or the like. Alternatively, the witness coupons may be directly written or deposited onto the component via a direct-write process, where the alloy is directly deposited onto the component via a melt-solidification process, physical vapor deposition, chemical vapor deposition, or the like. In addition, the alloy and/or pure element powder mixtures may be written onto the component and then be subsequently heat-treated at suitable temperatures. The deposited powder may also be treated by electron beam welding, or by a laser cladding process, or the like.

During operation, high temperature gas turbine components typically experience high operating temperatures for a given period of time, and are then shut down. The shut down process is similar to a quenching process, where the component is quickly cooled down from the high operating temperature. These alloy-based witness coupons are designed so that the time spent at the high operating temperature changes the metallurgical characteristics of the alloy in the alloy-based witness coupon, and the fast cooling of the component and the alloy-based witness coupon during the shut down process preserves the high temperature microstructure in the alloy-based witness coupon. The metallurgical characteristic change in the alloy-based witness coupon can allow the last temperature and/or the average temperature of a high temperature gas turbine component to be accurately estimated therefrom. The diffusion couple witness coupons are designed so that the time spent at the high operating temperature causes the pure elements and/or alloys to diffuse into one another in varying degrees. These diffusion characteristic changes can allow the average temperature of a high temperature gas turbine component to be accurately estimated therefrom.

Which temperature is estimated depends on the diffusion process of the elements in the alloy, and the time the component spends in operation. For example, if the diffusion process is fast, such as in the Co—Al two-phase alloy-based witness coupon system, and the component spends a long time in operation, the temperature estimated is the last temperature experienced by the component. If the diffusion process is slow, such as in the Co—Pt two-phase alloy-based witness coupon system, and the component spends a relatively short amount of time in operation (i.e., less than about 200 hours), the temperature estimated is the average temperature experienced by the component. In the case of the diffusion couple witness coupons, the estimated temperature is always the average temperature that is experienced by the component.

In the alloy-based witness coupons, the exposure or operating temperatures of the turbine components can be estimated by analyzing the phase formation of these witness coupons. During high temperature operation, the alloy in the alloy-based witness coupons is designed to experience metallurgical changes (i.e., phase fraction, lattice parameter, etc.) according to various temperatures. The change of the metallurgical characteristics allows the exposure or operating temperatures of the alloy-based witness coupons to be evaluated. Since the time that a component is in operation is known, the operating temperatures can be back-calculated from the time and microstructural or metallurgical changes that are observed in the alloy-based witness coupons.

In the diffusion couple witness coupons, the exposure or operating temperatures of the turbine components can be estimated by analyzing the diffusion kinetics of these diffusion couple witness coupons. During high temperature operation, the pure elements and/or alloys in the diffusion couple witness coupons are designed to interact and diffuse into one another to form intermetallic compounds or interdiffusion zones according to various temperatures. The formation of these intermetallic compounds or interdiffusion zones, as well as the thickness of the zones, allows the exposure or operating temperatures of the diffusion couple witness coupons to be evaluated. An SEM micrograph showing the interdiffusion 50 between the rhodium (Rh) 30 and the platinum (Pt) 40 in one diffusion couple witness coupon is shown in FIG. 2. Since the time that a component is in operation is known, the operating temperatures can be back-calculated from the diffusion characteristic changes that are observed in the diffusion couple witness coupons.

During a shut down of the turbine, or at any other suitable time, the witness coupons may be removed from the component, and the phase formation and/or diffusion kinetics of the witness coupon can be analyzed. Alternatively, the witness coupons may be analyzed while still on, or attached to, the component. The analysis may be done either destructively (i.e., via microprobe analysis or nanoindentation, etc.) or non-destructively (i.e., via x-ray diffraction or neutron diffraction analysis, etc.). Preferably, if the witness coupon is still attached to the component when the analysis is performed, the analysis will be non-destructive to the component itself.

For example, the chemistry or composition of the alloy-based witness coupons could be determined by electron microprobe analysis. The diffusion profiles that are measured from electron microprobe analysis allow the temperature to be calculated based on the diffusion coefficients of the materials in the alloy-based witness coupons. While this is a destructive process to the alloy-based witness coupons, it is non-destructive to the turbine component itself.

The lattice parameter/phase fraction of the alloy-based witness coupons could be determined by x-ray diffraction analysis or by neutron diffraction analysis. This process is a non-destructive process, both for the alloy-based witness coupons and the turbine component itself.

The hardness/modulus of either the alloy-based witness coupons or the diffusion couple witness coupons could be determined by nanoindentation. While this is a destructive process to the witness coupons, it is non-destructive to the turbine component itself.

The micro-voltage of either the alloy-based witness coupons or the diffusion couple witness coupons could be determined by thermoelectric unit measurements. This measurement is based on the thermoelectric principle known as the Seebeck effect. This process is a non-destructive process, both for the witness coupons and the turbine component itself.

The diffusion characteristics of the diffusion couple witness coupons could be determined by electron microprobe analysis using either wavelength dispersive spectroscopy (WDS) or energy dispersive spectroscopy (EDS), or nanoindentation. While this is a destructive process to the diffusion couple witness coupons, it is non-destructive to the turbine component itself.

The results of such analyses are representative of the crystalline structure and chemical composition of the metal or alloy being tested. The results of the analyzed witness coupons may then be compared to the calibrated and catalogued witness coupons, and the operating temperatures of the witness coupons may be estimated therefrom based on the changes of the temperature-sensitive characteristics. Since the witness coupons experience the same temperature cycles as the turbine component it is attached to, the operating temperatures of the component can therefore be obtained from the estimated temperatures that the witness coupons experienced. Thereafter, since the time that the component has spent in operation is known, the remaining operating life and/or accumulated damage of the component can be determined based on the temperature estimations.

The alloy-based witness coupons may comprise any suitable alloys that comprise at least two phases and have the characteristic that the solubility of at least one element in one of the phases changes greatly with temperature changes, such as, for example, Co—Cr (where the Cr content is about 65-80 wt. %), Pt—Cr (where the Cr content is about 57-80 wt. %), or Co—Al (where the Al content is about 8-18 wt.

The diffusion couple witness coupons preferably comprise precious metals and/or their alloys, as well as an oxidation-resistant alloy comprising aluminum, chromium, silicon, titanium, etc. Precious metals are preferred because they have outstanding oxidation and hot-corrosion resistance, both of which are necessary to survive the harsh, high temperature environment in which turbines operate. Coating materials comprising NiAl, Ni(Pt)Al, and MCrAlY, where M stands for Ni, Co or Fe, also exhibit outstanding oxidation and hot-corrosion resistance, and they are also suitable as components of the diffusion couple witness coupons. The thickness of the diffusion couples depends on the temperature and time that a particular high temperature component experiences. The thickness should be sufficient so that complete homogenization of the compositions does not occur.

Embodiments of this invention comprise methods for estimating the temperatures that a high temperature component has experienced so that the remaining operational life of the high temperature component can be assessed. In one embodiment, the method comprises: attaching a witness coupon to the high temperature component; allowing the component and the witness coupon to cycle through at least one cycle of high temperature operation and shut down; analyzing the witness coupon; and estimating the temperatures that the high temperature component has experienced based on the data acquired by analyzing the witness coupon. This method may further comprise the steps of: removing the witness coupon from the high temperature component prior to analyzing the witness coupon, estimating the remaining operational life of the high temperature component, and/or estimating the accumulated damage to the high temperature component.

The attaching step may comprise the following steps, or means for performing the following steps: mechanically attaching the witness coupon onto the high temperature component, diffusion bonding the witness coupon onto the high temperature component, electron beam welding the witness coupon onto the high temperature component, laser welding the witness coupon onto the high temperature component, brazing the witness coupon onto the high temperature component, spraying the witness coupon onto the high temperature component, sputtering the witness coupon onto the high temperature component, ion plasma processing the witness coupon onto the high temperature component, directly depositing the witness coupon onto the high temperature component via a melt-solidification process, directly depositing the witness coupon onto the high temperature component via physical vapor deposition, directly depositing the witness coupon onto the high temperature component via chemical vapor deposition, depositing the witness coupon onto the high temperature component via a laser cladding process, depositing the witness coupon onto the high temperature component via an electron cladding process, depositing a powder onto the high temperature component followed by consolidation of the powder via sintering, depositing a paste onto the high temperature component followed by consolidation of the paste via sintering, and/or depositing a tape onto the high temperature component followed by consolidation of the tape via sintering.

The analyzing step may comprise the following steps, or means for performing the following steps: electron microprobe analysis using either wavelength dispersive spectroscopy or energy dispersive spectroscopy, x-ray fluorescence, laser plasma spectroscopy, high energy x-ray, neutron diffraction analysis, image analysis integrating optical microscopy, image analysis integrating optical or electron microscopy, nanoindentation, microhardness testing, ultrasonic modulus measurement techniques, eddy current probing, and/or thermoelectric unit measurements.

The witness coupons of this invention may comprise alloy-based witness coupons or diffusion couple witness coupons. The alloy-based witness coupons may comprise: an alloy comprising at least two phases, a binary alloy comprising cobalt and chromium, a binary alloy comprising platinum and chromium, a binary alloy comprising cobalt and aluminum, and/or a binary alloy comprising a precious metal. The diffusion couple witness coupon may comprise: Co30CrCo20Cr10Al (weight %), Pt—Co30Cr (weight %), rhodium, platinum, palladium, a precious metal, and/or an alloy of a precious metal. The diffusion couple witness coupon may further comprise a coating comprised of: NiAl, Ni(Pt)Al, and/or MCrAlY, where M stands for Ni, Co or Fe as members of the couples or as a protective coating of the diffusion couples.

Embodiments of this invention also comprise systems for estimating the temperatures that a high temperature component has experienced so that the remaining operational life of the high temperature component can be assessed. In one embodiment, these systems comprise: a means for attaching a witness coupon to the high temperature component; a means for allowing the component and the witness coupon to cycle through at least one cycle of high temperature operation and shut down; a means for analyzing the witness coupon; and a means for estimating the temperatures that the high temperature component has experienced based on the data acquired by analyzing the witness coupon. These systems may further comprise: a means for removing the witness coupon from the high temperature component prior to analyzing the witness coupon, a means for estimating the remaining operational life of the high temperature component, and/or a means for estimating the accumulated damage to the high temperature component.

Embodiments of this invention also comprise methods of making a witness coupon. In one embodiment, the method may comprise: casting an alloy ingot of an alloy-based witness coupon using induction melting, arc melting, or the like; performing high temperature annealing to homogenize the alloy ingot; and sectioning the alloy ingot into appropriate sized alloy-based witness coupons that can be attached to a high temperature component. In another embodiment, the method may comprise: depositing a layer of a material onto a high temperature component using micro-pen direct-write method, physical vapor deposition, chemical vapor deposition, or the like; depositing additional layers of the material onto the high temperature component as desired to make an alloy-based witness coupon. In yet another embodiment, the method may comprise: pressing thin foils of metals together using cold pressing, cold isostatic pressing, hot isostatic pressing, or the like to make diffusion couple sheets; sectioning the diffusion couple sheets into appropriate sized diffusion couple witness coupons that can be attached to a high temperature component.

As described above, the systems and methods of the present invention allow the remaining operational life of gas turbine components to be easily, accurately, conveniently and reliably assessed. Advantageously, these systems and methods are also non-destructive to the turbine components themselves.

Various embodiments of the invention have been described in fulfillment of the various needs that the invention meets. It should be recognized that these embodiments are merely illustrative of the principles of various embodiments of the present invention. Numerous modifications and adaptations thereof will be apparent to those skilled in the art without departing from the spirit and scope of the present invention. For example, while this invention has been described in terms of witness coupons for use in gas turbines, numerous other applications of these witness coupons are possible—for example, these witness coupons may be useful to other applications involving a hostile environment and/or rotating parts. Thus, it is intended that the present invention cover all suitable modifications and variations as come within the scope of the appended claims and their equivalents.

What is claimed is:

1. A method for estimating the temperatures that a high temperature component has experienced so that the remaining operational life and the accumulated damage of the high temperature component can be assessed, the method comprising:
attaching a witness coupon to the high temperature component;
allowing the component and the witness coupon to cycle through at least one cycle of high temperature operation and shut down;
analyzing the witness coupon; and
estimating the temperatures that the high temperature component has experienced based on the data acquired by analyzing the witness coupon.

2. The method of claim 1, wherein the attaching step comprises at least one of: mechanically attaching the witness coupon onto the high temperature component, diffusion bonding the witness coupon onto the high temperature component, electron beam welding the witness coupon onto the high temperature component, laser welding the witness coupon onto the high temperature component, brazing the witness coupon onto the high temperature component, spraying the witness coupon onto the high temperature component, sputtering the witness coupon onto the high temperature component, ion plasma processing the witness coupon onto the high temperature component, directly depositing the witness coupon onto the high temperature component via a melt-solidification process, directly depositing the witness coupon onto the high temperature component via physical vapor deposition, directly depositing the witness coupon onto the high temperature component via chemical vapor deposition, depositing the witness coupon onto the high temperature component via a laser cladding process, depositing the witness coupon onto the high temperature component via an electron cladding process, depositing a powder onto the high temperature component followed by consolidation of the powder via sintering, depositing a paste onto the high temperature component followed by consolidation of the paste via sintering, and depositing a tape onto the high temperature component followed by consolidation of the tape via sintering.

3. The method of claim 1, further comprising:
removing the witness coupon from the high temperature component prior to analyzing the witness coupon.

4. The method of claim 1, wherein the analyzing step comprises at least one of: electron microprobe analysis using at least one of wavelength dispersive spectroscopy and energy dispersive spectroscopy; x-ray fluorescence; laser plasma spectroscopy; high energy x-ray; neutron diffraction analysis; image analysis integrating optical microscopy; image analysis integrating optical microscopy; image analysis integrating electron microscopy; nanoindentation; microhardness testing; ultrasonic modulus measurement techniques; eddy current probing; and thermoelectric unit measurements.

5. The method of claim 3, wherein the analyzing step comprises at least one of: electron microprobe analysis using at least one of wavelength dispersive spectroscopy and energy dispersive spectroscopy; x-ray fluorescence; laser plasma spectroscopy; high energy x-ray; neutron diffraction analysis; image analysis integrating optical microscopy; image analysis integrating optical microscopy; image analysis integrating electron microscopy; nanoindentation; microhardness testing; ultrasonic modulus measurement techniques; eddy current probing; and thermoelectric unit measurements.

6. The method of claim 1, further comprising:
estimating the remaining operational life of the high temperature component.

7. The method of claim 1, further comprising:
estimating the accumulated damage to the high temperature component.

8. The method of claim 1, wherein the witness coupon comprises an alloy-based witness coupon.

9. The method of claim 8, wherein the alloy-based witness coupon comprises at least one of: an alloy comprising at least two phases, a binary alloy comprising cobalt and chromium, a binary alloy comprising platinum and chromium, a binary alloy comprising cobalt and aluminum, and a binary alloy comprising a precious metal.

10. The method of claim 1, wherein the witness coupon comprises a diffusion couple witness coupon.

11. The method of claim 10, wherein the diffusion couple witness coupon comprises at least one of: Co30Cr—Co20Cr10Al (weight %), Pt—Co30Cr (weight %), rhodium, platinum, palladium, a precious metal, and an alloy of a precious metal.

12. The method of claim 10, wherein the diffusion couple witness coupon further comprises a protective coating.

13. The method of claim 12, wherein the coating comprises at least one of: NiAl, Ni(Pt)Al, and MCrAlY, where M stands for Ni, Co or Fe.

14. A system for estimating the temperatures that a high temperature component has experienced so that the remaining operational life and the accumulated damage of the high temperature component can be assessed, the system comprising:
a means for attaching a witness coupon to the high temperature component;
a means for allowing the component and the witness coupon to cycle through at least one cycle of high temperature operation and shut down;
a means for analyzing the witness coupon; and
a means for estimating the temperatures that the high temperature component has experienced based on the data acquired by analyzing the witness coupon.

15. The system of claim 14, wherein the means for attaching a witness coupon to the high temperature component comprises at least one of: a means for mechanically attaching the witness coupon onto the high temperature component, a means for diffusion bonding the witness coupon onto the high temperature component, a means for electron beam welding the witness coupon onto the high temperature component, a means for laser welding the witness coupon onto the high temperature component, a means for brazing the witness coupon onto the high temperature component, a means for spraying the witness coupon onto the high temperature component, a means for sputtering the witness coupon onto the high temperature component, a means for ion plasma processing the witness coupon onto the high temperature component, a means for directly depositing the witness coupon onto the high temperature component via a melt-solidification process, a means for directly depositing the witness coupon onto the high temperature component via physical vapor deposition, a means for directly depositing the witness coupon onto the high temperature component via chemical vapor deposition, a means for depositing the witness coupon onto the high temperature component via a laser cladding process, a means for depositing the witness coupon onto the high temperature component via an electron cladding process, a means for depositing a powder onto the high temperature component followed by consolidation of the powder via sintering, a means for depositing a paste onto the high temperature component followed by consolidation of the paste via sintering, and a means for depositing a tape onto the high temperature component followed by consolidation of the tape via sintering.

16. The system of claim 14, further comprising:
a means for removing the witness coupon from the high temperature component prior to analyzing the witness coupon.

17. The system of claim 14, wherein the means for analyzing the witness coupon comprises at least one of: wavelength dispersive spectroscopy and energy dispersive spectroscopy; x-ray fluorescence; laser plasma spectroscopy; high energy x-ray; neutron diffraction analysis; image analysis integrating optical microscopy; image analysis integrating optical microscopy; image analysis integrating electron microscopy; nanoindentation; microhardness testing; ultrasonic modulus measurement techniques; eddy current probing; and thermoelectric unit measurements.

18. The system of claim 16, wherein the means for analyzing the witness coupon comprises at least one of: wavelength dispersive spectroscopy and energy dispersive spectroscopy; x-ray fluorescence; laser plasma spectroscopy; high energy x-ray; neutron diffraction analysis; image analysis integrating optical microscopy; image analysis integrating optical microscopy; image analysis integrating electron microscopy; nanoindentation; microhardness testing; ultrasonic modulus measurement techniques; eddy current probing; and thermoelectric unit measurements.

19. The system of claim 14, further comprising:
a means for estimating the remaining operational life of the high temperature component.

20. The system of claim 14, further comprising:
a means for estimating the accumulated damage to the high temperature component.

21. The system of claim 14, wherein the witness coupon comprises an alloy-based witness coupon.

22. The system of claim 21, wherein the alloy-based witness coupon comprises at least one of: an alloy comprising at least two phases, a binary alloy comprising cobalt and chromium, a binary alloy comprising platinum and chromium, a binary alloy comprising cobalt and aluminum, and a binary alloy comprising a precious metal.

23. The system of claim 14, wherein the witness coupon comprises a diffusion couple witness coupon.

24. The system of claim 23, wherein the diffusion couple witness coupon comprises at least one of: Co30Cr—Co20Cr10Al (weight %), Pt—Co30Cr (weight %), rhodium, platinum, palladium, a precious metal, and an alloy of a precious metal.

25. The system of claim 23, wherein the diffusion couple witness coupon further comprises a protective coating.

26. The system of claim 25, wherein the coating comprises at least one of: NiAl, Ni(Pt)Al, and MCrAlY, where M stands for Ni, Co or Fe.

27. A method of making an alloy-based witness coupon, the method comprising the steps of:
casting an alloy ingot;
annealing the cast alloy ingot to homogenize the cast alloy ingot; and
sectioning the annealed cast alloy ingot into predetermined sized sections to form the alloy-based witness coupons.

28. The method of claim 27, wherein the casting step comprises at least one of: induction melting and arc melting.

29. The method of claim 27, wherein the alloy-based witness coupon comprises at least one of: an alloy comprising at least two phases, a binary alloy comprising cobalt and chromium, a binary alloy comprising platinum and chromium, a binary alloy comprising cobalt and aluminum, and a binary alloy comprising a precious metal.

30. A method of making an alloy-based witness coupon, the method comprising the steps of:
depositing a layer of a material onto a high temperature component; and
repeating the depositing step as many times as necessary to create the desired alloy-based witness coupon.

31. The method of claim 30, wherein the depositing layer comprises at least one of: directly writing the material onto the high temperature component, physical vapor deposition of the material onto the high temperature component, chemical vapor deposition of the material onto the high temperature component, sputtering the material onto the high temperature component, thermal spraying the material onto the high temperature component, ion plasma deposition of the material onto the high temperature component, applying a paste of the material onto the high temperature component followed by consolidation of the material via sintering, applying a tape comprising the material onto the high temperature component followed by consolidation of the material via sintering, powder deposition of the material onto the high temperature component followed by consolidation of the material via sintering, laser cladding the material onto the high temperature component, and electron cladding the material onto the high temperature component.

32. The method of claim 30, wherein the alloy-based witness coupon comprises at least one of: an alloy comprising at least two phases, a binary alloy comprising cobalt and chromium, a binary alloy comprising platinum and chromium, a binary alloy comprising cobalt and aluminum, and a binary alloy comprising a precious metal.

33. A method of making a diffusion couple witness coupon, the method comprising the steps of:
placing two metal foils together;
joining the two metal foils together; and
sectioning the joined metal foils into predetermined sized sections to form one or more diffusion couple witness coupons.

34. The method of claim 33, wherein the joining step comprises at least one of: welding the edges of the two metal foils together, hot isostatically pressing the two metal foils together, and cold isostatically pressing the two metal foils together.

35. The method of claim 33, wherein the diffusion couple witness coupon comprises at least one of: Co30Cr—Co20Cr10Al (weight %), Pt—Co30Cr (weight %), rhodium, platinum, palladium, a precious metal, and an alloy of a precious metal.

36. The method of claim 33, wherein the diffusion couple witness coupon comprises a protective coating.

37. The method of claim 36, wherein the coating comprises at least one of:
NiAl, Ni(Pt)Al, and MCrAlY, where M stands for Ni, Co or Fe.

38. A method for estimating the temperatures that a high temperature component has experienced so that the remaining operational life and the accumulated damage of the high temperature component can be assessed, the method comprising:
attaching an alloy-based witness coupon to the high temperature component;
allowing the component and the alloy-based witness coupon to cycle through at least one cycle of high temperature operation and shut down;
analyzing the alloy-based witness coupon; and
estimating the temperatures that the high temperature component has experienced based on the data acquired by analyzing the alloy-based witness coupon, wherein the alloy-based witness coupon comprises an alloy comprising at least two phases wherein the solubility of at least one element in one of the phases changes greatly with temperature changes.

39. A method for estimating the temperatures that a high temperature component has experienced so that the remaining operational life and the accumulated damage of the high temperature component can be assessed, the method comprising:
attaching a diffusion couple witness coupon to the high temperature component;
allowing the component and the diffusion couple witness coupon to cycle through at least one cycle of high temperature operation and shut down;
analyzing the diffusion couple witness coupon; and
estimating the temperatures that the high temperature component has experienced based on the data acquired by analyzing the diffusion couple witness coupon.
